(19) United States
(12) Patent Application Publication
(10) Pub. No.: US 2004/0248300 A1
(43) Pub. Date: Dec. 9, 2004

(54) Enhanced gene expression using vectors

(76) Inventor: Christopher Maurice Preston, Glasgow (GB)

Correspondence Address: Daniel A Monaco, Drinkler Biddle & Reath, One Logan Square, 18th & Cherry Streets, Philadelphia, PA, 19103-6996, US

(21) Appl. No.: 10/489,744

(22) PCT Filed: Jul. 22, 2002
(86) PCT No.: PCT/GB02/03348

(30) Foreign Application Priority Data
Sep. 14, 2001 (GB) 0122232.2

(51) Int. Cl. C12N 15/86; C12N 7/00

(52) U.S. Cl. 435/456; 435/235.1

(57) ABSTRACT

There is described a vector able to express a transgene under the control of a promoter, the duration of expression being enhanced by the exposure of the vector to cytomegalovirus pp71 or a homologue thereof. Usually the vector will be a viral vector and Herpesvirus vectors are preferred. Suitable cytomegalovirus pp71 proteins include, but are not limited to, mouse, rat, chimpanzee, simian, equine and guinea pig pp71 proteins, but human pp71 is preferred. The vector may itself include the gene for expression of cytomegalovirus pp71.

The present invention relates to enhancing the duration of transgene expression in vectors.

Herpesviruses include Herpes Simplex Virus types 1 and 2 (HSV-1 and HSV-2), Human Cytomegalovirus (HCMV), Epstein-Barr Virus (EBV) and Equine Herpesviruses 1 and 4 (EHV-1 and EHV-4). The term “Herpesvirus” is used herein to refer to any virus of the herpesvirus family, including viruses in the α group (eg HSV-1 & 2, EHV 1 & 4), the β group (eg HCMV) and in the γ group (eg EBV).

Herpes simplex virus type 1 (HSV-1) is a human virus that establishes latency in sensory neurons. The latent state is maintained for the lifetime of an individual, although periodic episodes of reactivation, manifested as cutaneous lesions, may occur. The stability of the retention of HSV-1 in neurons suggests its use as a gene therapy vector, particularly for the treatment of neurological diseases. Conceptually, it should be possible to engineer the virus such that therapeutic gene products are produced from the otherwise silent latent genome. HSV-1, however, can cause fatal encephalitis in humans and, furthermore, each of the immediate early (IE) proteins is toxic to cells in culture. Therefore, candidate vectors must first be stringently disabled for replication and as impaired as possible for IE gene expression. A second requirement is to achieve long term expression of foreign genes, preferably with a means to control such genes. Prevention of IE gene expression has been achieved by the construction of the HSV-1 mutant in1312 (Preston et al., 1998). This virus contains a 12 base pair insertion in the coding sequences for VP16 (Ace et al., 1988; 1989), a deletion of the essential RING domain of ICP0 (Everett, 1987), and the temperature sensitive mutation of tsK that inactivates ICP4 (Davison et al., 1984). As a consequence of the three mutations, in1312 is extremely impaired for IE gene expression and has low cytotoxicity for cells in culture. The possibility of using an in1312-based virus as a vector is exemplified by the finding that a latency-active promoter, when cloned into in1312, remains active in sensory neurons of mice (Marshall et al., 2000).

A problem that hinders the application of HSV-1 as a vector concerns the fact that, when IE gene expression is blocked, the genome becomes repressed and reaches a quiescent state in infected cells (Preston and Nicholl, 1997; Samaniego et al., 1998). In the quiescent state, promoters cloned into the HSV-1 are not active or responsive to trans-acting factors, and thus they cannot be used to direct the long term expression of therapeutic foreign gene products. In addition, regulation of the promoter driving expression of the foreign gene cannot be achieved due to the repression.

It has now been found that an in1312-based mutant containing the human cytomegalovirus (HCMV) UL82 gene encoding the protein pp71, and also containing the Escherichia coli lacZ gene, which encodes β-galactosidase, controlled by the major HCMV IE promoter exhibits maintained expression. The HCMV IE promoter is a potent element for directing gene expression, but it is repressed in tissue culture when cloned into in1312. A control virus, in1382, contains just the HCMV IE-lacZ insertion. When cells are infected with in1382, the HCMV IE promoter is repressed and thus cells fail to contain β-galactosidase within approximately 4 days of infection. This failure to express gene products for extended periods in tissue culture is typical of all promoters tested to date. We show that inclusion of the pp71 coding sequences in the genome of in1312 enables the HCMV IE promoter to overcome repression, resulting in the production of β-galactosidase for extended periods in cultured human fibroblasts.

A further feature of in1312 is that the mutation in the IE regulatory protein ICP4 is temperature sensitive, thus the protein is active at 31° C. This activity of ICP4 is sufficient to drive plaque formation of in1312 at 31° C. (albeit at low efficiency due to the mutations in VP16 and ICP0). After approximately 4 days after infection with in1312 at 38.5° C., downshifting cultures to 31° C. does not result in any plaque formation, because the repression has rendered the genome insensitive to the stimulatory effect of ICP4. We show here that the pp71 coding sequences overcome this aspect of repression also, such that genomes remain responsive to ICP4 and are able to form plaques upon downshift.

HCMV is an important human pathogen, and the tegument protein pp71 has a significant role in the viral transcription programme since mutants deleted for pp71 coding sequences initiate productive infection inefficiently as low moi (Bresnahan and Shenk, 2000). At present there is little detailed information on the mechanism of action of pp71. The studies of Liu and Stinski (Liu et al., 1992) suggested that the protein acts through the cellular ATF and/or AP-1 transcription factors in transfection assays. Our findings, however, point to a less stringent specificity since a range of promoters, not necessarily containing ATF/AP-1 binding sites, are responsive to the protein in the context of the HSV-1 genome (Homer et al., 1999).

The present invention provides recombinant constructs for expression of a transgene, comprising:

a) a first vector comprising a promoter operably linked to said transgene; and

b) a gene for cytomegalovirus pp71 or homologues thereof in expressible form.

The gene for cytomegalovirus pp71 or a homologue thereof may be provided as part of a second vector able to express cytomegalovirus pp71 or its homologue. Alternatively the gene for cytomegalovirus pp71 or a homologue thereof may be integrated into a host cell genome, the host cell then being transfected with the first vector containing the transgene of interest. Finally, the gene for cytomegalovirus pp71 or a homologue thereof may be included as a component of the first vector.

The first vector will usually be a viral vector and is preferably a non-integrating viral vector (ie. a viral vector which does not integrate into the host cell genome). Suitably the vector is a Herpesvirus vector. By the term “Herpesvirus vector” we mean that the vector is derived from or is a genetically manipulated version of a naturally occurring Herpesvirus.

The first vector may however be a viral vector based upon any suitable virus. Thus, any virus which does not integrate into the genome of the host cell could be used to form a suitable vector. Mention may be made of Herpesviruses, especially HSV-1 and HSV-2, and defective HSV-1 and HSV-2 vectors otherwise known as amplicons. Also suitable is adenovirus.

The second vector, where used, may be based upon any vector able to express the cytomegalovirus pp71 gene within the host cell. Any suitable promoter may be used to drive expression.

Suitable host cells will depend upon the first vector selected for use, and there is a vast amount of information available to those skilled in the art on the selection of host cells. The invention encompasses animal host cells, and preferred host cells include mammalian host cells. It should be noted that HSV can infect almost all mammalian cells and hence there is very little limitation of host cell type using an HSV based first vector.

The promoter used in the first vector may be any promoter capable of achieving the required level of expression of the transgene. A viral promoter may be used and an example of suitable promoters includes the HSV-1 IE promoter, the HCMV IE promoter or equivalent IE promoters from related viruses. Optionally, the promoter may drive both the transgene and pp71 where both are present on the first vector, but this is not essential and separate promoters for each gene may be preferable in some instances.

Optionally the transgene and pp71 may be juxataposed together within the first vector, but this is not essential for the enhancement of expression. All that is required when both elements are present on the first vector is that both elements are inserted into the first vector such that no essential cis-acting sequence is disrupted.

pp71 of any cytomegalovirus may be used in the invention, but those of particular interest are pp71 from the human, mouse, rat, chimpanzee, simian, equine and guinea pig cytomegaloviruses. Human cytomegalovirus pp71 is preferred. The reference to “homologues” refers to equivalent proteins to pp71 that may exist in other virus types and to slight modifications of such genes as described hereinafter.

The gene for cytomegalovirus pp71 which is central to the continued expression of the transgene preferably comprises the whole of the gene sequence. However, slight modifications to the gene sequence will occur in different naturally occurring variants of the cytomegalovirus without affecting the effect of cytomegalovirus pp71 on the vector and similar modifications, even if deliberately introduced, may therefore be likewise tolerated. Further, small deletions of the gene sequences (usually 1 or 2%, but including deletions of up to 5% or 10%, and possibly as high as 30%) may also not affect the function of the pp71 gene in the vector function and are also comprised by the term “homologues”. In general however the homologues referred to herein exhibit at least 70% homology, preferably 80% homology or above with the naturally occurring sequence of a cytomegalovirus pp71 gene. More desirably the homologues referred to herein will have 85% or more, for example 90% or more homology with the naturally occurring sequence of a cytomegalovirus pp71 gene. Most preferably the homologues referred to herein will have 92, 93, 94, 95, 96, 97, 98 or 99% homology with the naturally occurring sequence of a cytomegalovirus pp71 gene.

The transgene may be any suitable gene sequence and will normally encode a protein or polypeptide of therapeutic value. However, the transgene might also encode an antisense RNA or ribozyme which provides therapeutic value by inactivating a host gene product. Whilst limitation of the possible transgenes able to be inserted into the first vector is not intended, mention may be made of peptide hormones (insulin, ACTH, vasopressin), growth factors, enzymes, and the like. The size of the transgene may determine the vector selected, for example a transgene of up to 15 kilobase pairs may be accommodated by HSV-1. However with a larger transgene insert the ability to replicate may be lost. Hence a larger transgene (up to a size of 150 kilobase pairs) could be inserted into an amplicon vector, which may then be accompanied by a helper virus to assist replication (see, for example, Spaete et al., 1982).

In a preferred embodiment expression of the transgene is regulatable by external factors. These may be physical (eg heat) or trans-acting factors. Regulation by external factors, such as drug administration, is of especial interest where the vector is to be administered to a patient in vivo.

In a further embodiment, the present invention provides a method of maintaining expression of a transgene in a vector, said method comprising introducing the gene for cytomegalovirus pp71 into the vector whilst maintaining the functionability of any cis-acting sequence.

In another embodiment, the present invention provides a method of maintaining expression of a transgene in a vector, said method comprising providing cytomegalovirus pp71 during expression of said transgene.

In a further aspect, the present invention provides a use for cytomegalovirus pp71 to promote the maintained expression of a transgene in a vector, usually a viral vector.

Additionally, the present invention provides a host cell infected with recombinant constructs as described above. The host cell may be cultured and infected in vitro and, optionally, may be introduced into a patient once transfection has been established as successful. Alternatively the vector itself may be introduced directly to a patient so that transfection occurs in vivo. Whilst the patient may be a human, in vivo infection of animals is also contemplated. Optionally the transgene may encode for a peptide which is required in pure or semi-pure form. The first vector may be used to produce said protein which may then be harvested from the host cell(s) and purified.

In a yet further embodiment, the invention provides a method of producing a target protein or peptide, said method comprising providing a first vector comprising a promoter operably linked to a transgene encoding the target protein or peptide and wherein during expression of said transgene the transgene is exposed to cytomegalovirus pp71 or a homologue thereof. In a preferred embodiment the first vector further comprises the gene for cytomegalovirus pp71 or a homologue thereof.

In a yet further aspect the present invention provides a method of treating a patient having a disease or disorder (for example a neurological disease or disorder), said method comprising introducing to said patient recombinant constructs as described above, wherein said constructs comprises a transgene encoding a substance (RNA or protein) of therapeutic value for said disease or disorder.

The present invention will now be further described with reference to the following non-limiting examples and figures in which:

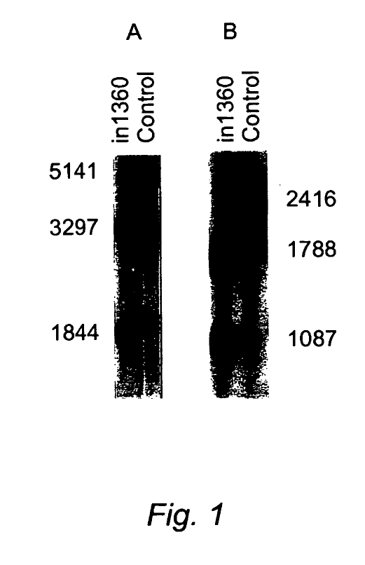

FIG. 1. Structure of in1360. Southern blots of in1360 and a control isolate that contained non-recombinant are shown. A shows an EcoRI plus BamHI plus HindIII digest, probed with radiolabelled p35. The sizes of the bands are shown to the left. The 5141 base pair band is derived from the normal UL43 locus, whereas the 3297 and 1844 base pair bands are formed as a consequence of the HCMV IE-lacZ insertion. B shows an EcoRI digest, probed with radiolabelled 2416 base pair fragment encompassing the TK coding sequences. The 2416 base pair fragment is derived from the normal TK locus, whereas the 1788 and 1087 base pair fragments are formed as a consequence of the HCMV IE-pp71 insertion.

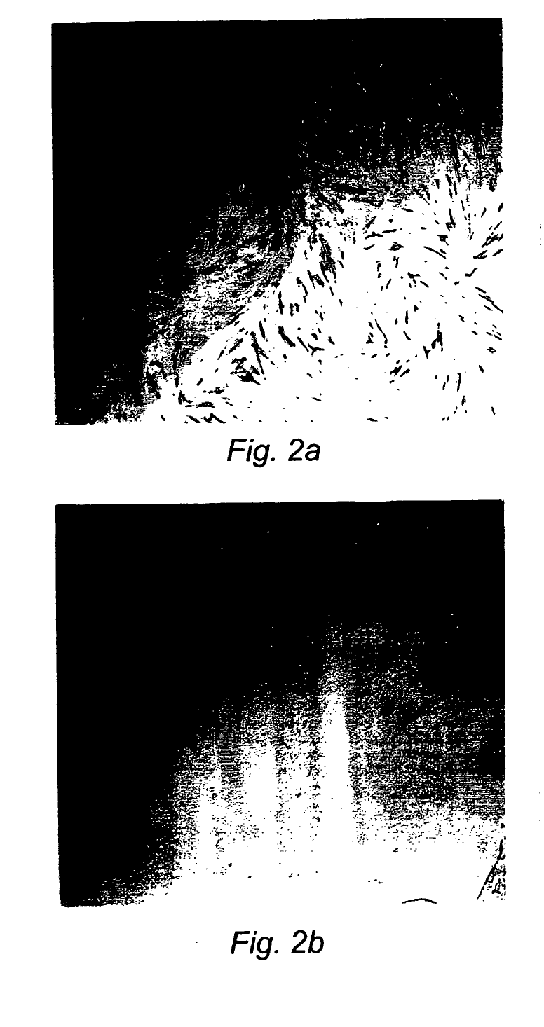

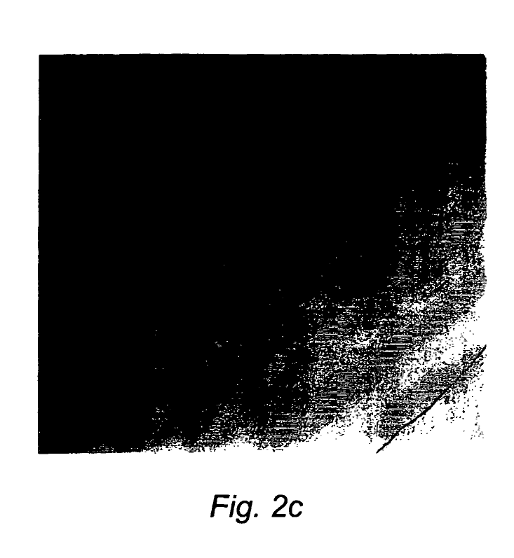

FIG. 2. Photographs of cells fixed and stained with X-gal, taken at 10 days after infection with in1360 (a), in1382 (b), or mock infected (c).

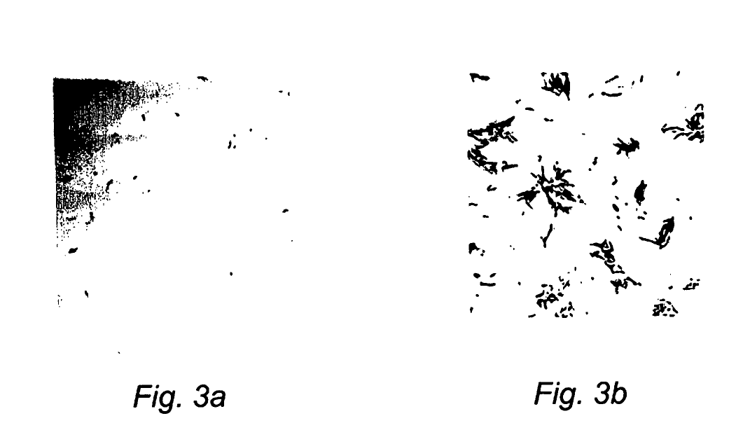

FIG. 3. Plaque formation upon downshift of in1360 infected cultures at 38.5° C. (a) and 31° C. (b).

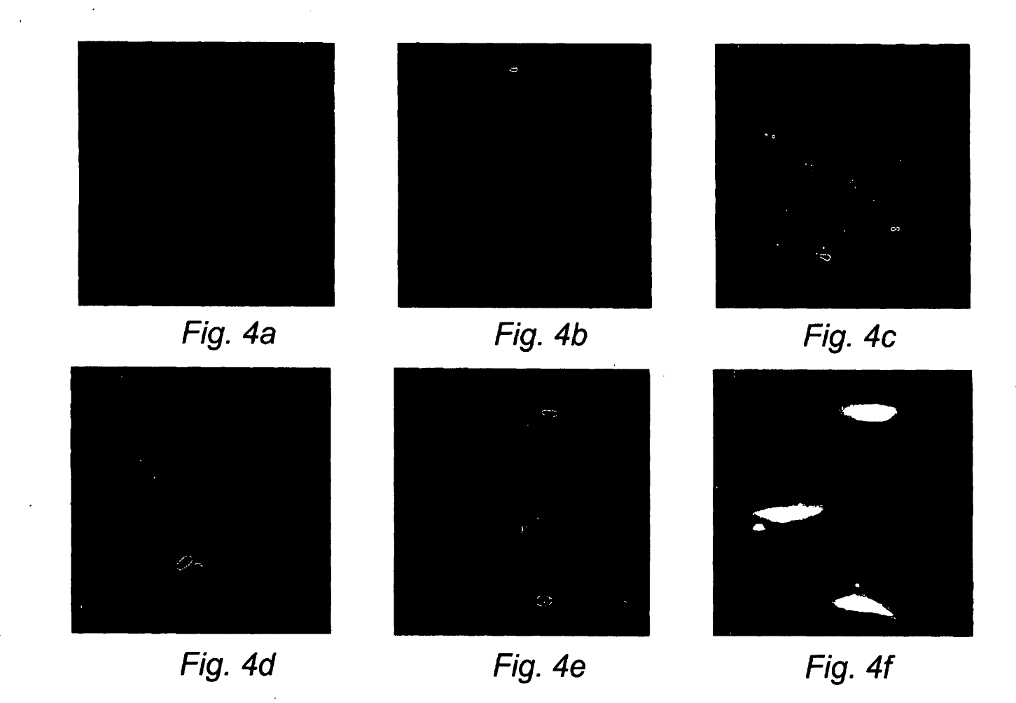

FIG. 4. Protein expressed in in1360 infected cells at 10 days post infection.

a. Anti β-galactosidase mouse mAb.

b. Anti-pp71 rabbit polyclonal antibody.

c. Yellow Fluorescent Protein (YFP)-pp71 fusion protein expressed.

d. Anti HSV-1 ICP4 mouse mAb.

e. Anti HSV-1 ICP27 mouse mAb.

f. Green Fluorescent Protein (GFP) expressed.

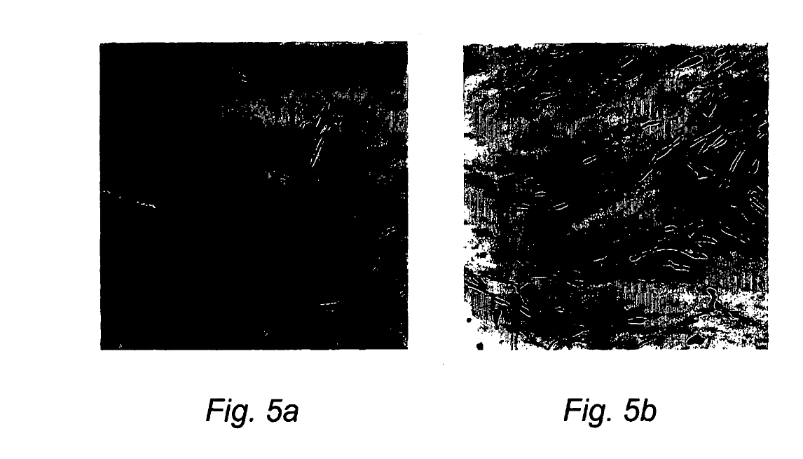

FIG. 5. Photographs of cells fixed and stained with X-gal, taken at 10 days after infection with in1360. On day 8, cells were untreated (a) or treated with 660 nM TSA (b).

### EXAMPLES

### Methods

Plasmids

Plasmid pCP376 contains the HCMV IE promoter driving expression of lacZ, inserted into the coding sequences for the HSV-1 UL43 gene. The starting point was p35, which is identical to pC75 (MacLean et al., 1991) except that the unique XbaI site was removed by treatment with Klenow enzyme and subsequent relegation. Plasmid p35 was provided by Dr C A MacLean (MRC Virology Unit, Glasgow). Plasmid p35 was cleaved with NsiI, which interrupts the UL43 coding sequences, treated with Klenow enzyme, and ligated with a 30 base pair double stranded oligonucleotide that contains sites for the restriction enzymes XhoI, BglII and XbaI. An isolate containing the oligonucleotide insert was isolated and named pCP99429. This plasmid was cleaved with XhoI and XbaI, and ligated with an XhoI/XbaI fragment containing the HCMV IE promoter plus lacZ sequences isolated from pMJ101 (Jamieson et al., 1995). A plasmid containing the HCMV IE-lacZ insert in pCP99429 was purified and named pCP376.

Viruses

The parental HSV-1 mutant was in1312. Mutant in1382 is a control virus, consisting of in1312 with the HCMV IE-lacZ construct inserted in the thymidine kinase (TK) coding sequences (Everett et al., 1988). Mutant in1324 is in1312 with the HCMV pp71 coding sequences, controlled by the HCMV IE promoter, inserted in the TK coding sequences (Homer et al., 1999). Mutant in1374 is in1312 with HCMV IE-lacZ inserted in the UL43 coding sequences. This mutant was produced by cotransfection of BHK-21 cells with in1312 DNA plus ScaI-cleaved pCP376. Virus isolates that gave blue plaques when incubated with 5-bromo-4-chloro-3-indolyl-β-D-galactoside (X-gal) were propagated, and DNA was prepared and tested by Southern hybridisation for the presence of the insert and the absence of parental in1312 DNA. An isolate that was pure was named in1374. Mutant in1360 contains the HCMV IE-pp71 insert in TK and the HCMV IE-lacZ insert in UL43. It was constructed by recombination between in1324 and in1374. BHK-21 cell monolayers were coinfected with the two mutants, maintained at 31° C. for 3 days, harvested and sonicated. Progeny virus was titrated on BHK-21 cells monolayers. Virus isolates that gave plaques that were both TK negative and blue when incubated with X-gal were purified and analysed by Southern hybridisation. An isolate that contained the HCMV IE-pp71 insert in TK and the HCMV IE-lacZ insert in UL43, with no detectable contamination from either parental virus, was named in1360 (FIG. 1). Virus titres are expressed as values on human osteosarcoma U2OS cells at 31° C. in the presence of HMBA: under these conditions the mutations of in1312 are overcome (Marshall et al., 2000).

### Example 1

Infection of Cells

Monolayers of human foetal foreskin fibroblasts (HFFF2) were infected with in1382 or in1360 and incubated at 38.5° C. in Dulbecco medium supplemented with 2% (v/v) foetal calf serum, 2 mM L-glutamine, 100 units/ml penicillin and 100 μg/ml streptomycin (DF2), with a change of medium every two days.

Histochemical Detection of β-galactosidase

Cells expressing β-galactosidase were detected by fixation in 1% (v/v) glutaraldehyde followed by staining with X-gal, as described by Jamieson et al., (1995).

Enzymatic Detection of β-galactosidase

Cell extracts were prepared and assayed for β-galactosidase using 4-methylumbellyferyl-β-D-galactoside, as described previously (Preston and Nicholl, 1997).

### Results

Construction of in1360

Southern blots of in1360 plaque isolates are shown (FIG. 1). To check for insertion of HCMV IE-lacZ in UL43 sequences, DNA from plaque isolates was cleaved with EcoRI, BamHI and HindIII and probed with radiolabelled p35. Genomes without insertions gave a single band of 5141 base pairs, whereas genomes with the insertion gave two bands of 3297 and 1844 base pairs. To check for insertion of HCMV IE-pp71 sequences, DNA from plaque isolates was cleaved with EcoRI and probed with a 2416 base pair EcoRI fragment that spans the site of insertion. Genomes without insertions gave a 2416 base pair fragment, whereas those with an insertion gave bands of 1788 and 1078 base pairs, as described previously (Rinaldi et al., 1999). The isolate indicated in FIG. 1 was designated in1360 since it contained no detectable DNA from either parent. A plaque isolate that was not pure, since it contained DNA from both of the viruses used in the recombination that yielded in1360 (the 5141 and 2416 base pair bands), is shown for comparison.

Expression of β-galactosidase in HFFF2 Cell Monolayers

Cell monolayers were infected with 10⁶ pfu of in1360 or in1382 and maintained at 38.5° C., with a change of culture medium every 2 days. From day 5 on, monolayers were harvested, the cells lysed, and extracts assayed for β-galactosidase. Results are shown in Table 1.

TABLE 1
Production of β-galactosidase in HFFF2 cells.

| | |
|---|---|
| | β-galactosidase |
| Days post | activity after infection |

| infection | in1360 | in1382 |
|---|---|---|
| 5 | 122 (4) | 14 (1) |
| 6 | 153 (18) | 9 (3) |
| 7 | 209 (7) | 7 (2) |
| 8 | 338 (10) | 4 (3) |
| 9 | 356 (4) | 13 (2) |
| 10 | 412 (8) | 6 (2) |

HFFF2 monolayers were infected with in1360 or in1382, and samples taken at various times for assay of β-galactosidase. Values given are fluorometric readings in arbitrary units, presented as the means of duplicate samples, with variation of individual measurements from the mean given in brackets. Values for mock infected cell monolayers were subtracted from all values presented. These were as follows: day 5, 4; day 6, 9; day 7, 8; day 8, 8; day 9, 6; day 10, 12.

In monolayers infected with in1382, enzyme activities were only marginally above those of mock infected cells. In monolayers infected with in1360, significant β-galactosidase activity was detected at five days post infection, and this level rose steadily during the next five days. Confirmation that monolayers infected with in1360 continued to express β-galactosidase was obtained by histochemical staining of cultures for the enzyme. As shown in FIG. 2, at 10 days post infection, many cells were positive for β-galactosidase after infection with in1360 (FIG. 2a), but no positive cells were detected in cultures infected with in1382 (FIG. 2b) or mock infected (FIG. 2c).

Continued Responsiveness to Downshift

Monolayers of HFFF2 cells were infected with 3×10⁴ pfu of in1360 or in1382 and maintained at 38.5° C. At 5, 7 and 9 days post infection, sample monolayers were overlaid with DF2 containing 2% (v/v) human serum, to prevent release and spread of virus, and maintained at 31° C. for 4 days. Monolayers were stained histochemically with X-gal and plaque numbers counted (Table 2).

TABLE 2
Formation of plaques upon temperature
downshift

| | |
|---|---|
| Time of Downshift | Number of Plaques |

| (days post infection) | in1360 | in1382 |
|---|---|---|
| 5 | 375 | 0 |
| 7 | 451 | 0 |
| 9 | 521 | 0 |

On cultures infected with in1360, downshift resulted in the formation of plaques, with the numbers increasing between 5 and 9 days post infection.

Cultures infected with in1382 showed no plaques when downshifted at 5, 7 or 9 days post infection.

### Discussion

The significance of the results is that expression of pp71 may enable foreign genes (transgenes) to be expressed from HSV-1 vectors for long periods, because repression is overcome. This would be an important step in vector development and would open the way for HSV-1 vectors to be used for long term, possibly permanent, repair of defects. In addition, the presence of pp71 coding sequences in vectors may enable regulation of expression of transgenes by drugs, so that the amount of therapeutic gene product made could be controlled. At present this is not possible because promoters become repressed and insensitive to regulation. The approach may not just be applicable to HSV-1 vectors. It may be useful for other herpesvirus vectors, or for other viral vectors.

### Example 2

Resumption of Replication Upon Downshift

To complement the data given in Table 2, photographs of in1360-infected cells after continued maintenance at 38.5° C. (FIG. 3a) or downshift to 31° C. (FIG. 3b) are presented. Monolayers of HFFF2 cells were infected with in1360 and maintained at 38.5° C. At 10 days after infection, monolayers were overlaid with medium containing 5% human serum, to prevent spread of released virus, and either maintained at 38.5° C. or downshifted to 31° C. After a further 3 days, monolayers were stained for β-galactosidase. Downshift to 31° C. results in the formation of plaques in the cell monolayer (see FIG. 3).

### Example 3

Expression of a Range of Proteins in1360-infected Cells

Example 1 described above shows the continued expression of β-galactosidase at 10 days post infection. The long term expression of other proteins was investigated by immunofluorescence. Monolayers of HFFF2 cells were infected with in1360 or derivatives and analysed at 10 days post infection. The results are shown in FIG. 4.

Panel a: An anti β-galactosidase mouse monoclonal antibody was used (obtained from Roche Diagnostics Corp., Roche Molecular Biochemicals, 9115 Hague Road, PO Box 50414, IN 46250-0414, USA, Catalogue No. 1083104).

Panel b: An anti-pp71 rabbit polyclonal antibody was used.

Panel c: Cells were infected with a derivative of in1360 that expresses a yellow fluorescent protein (YFP)-pp71 fusion protein instead of pp71.

Fluorescence of the YFP-pp71 fusion protein (molecular weight 97,000) was detected.

Panel d: A mouse monoclonal antibody that recognises the HSV-1 ICP4 immediate early protein was used.

Panel e: A mouse monoclonal antibody (obtained from AutogenBioclear, Holly Ditch Farm, Mile Elm, Calne, Wiltshire, SN11 0PY, United Kingdom, Catalogue No. 13-126-100) that recognises the HSV-1 ICP27 immediate early protein was used.

Panel f: Cells were infected with a derivative of in1360 that expresses green fluorescent protein (GFP) instead of β-galactosidase. Direct fluorescence of GFP (molecular weight 30,000) was detected in live cells. A monochromatic image is presented.

No fluorescent cells were observed after infection with the mutant in1382, which does not express pp71.

These experiments show that pp71 directs long term expression of a range of proteins, including itself. The effect described is not restricted to β-galactosidase.

### Example 4

The in1360 Genome Remains Responsive to Trichostatin A

Trichostatin A (TSA) is an agent that inhibits deacetylases. When it is added to cells, histones and other proteins involved in transcriptions become hyperacetylated, because they are not deacetylated. This frequently results in activation of gene expression. In the case of HSV-1 mutants, the genome is not responsive to TSA once the quiescent state has been established. Monolayers of HFFF2 cells were infected with in1360 and maintained at 38.5° C. At 8 days after infection, cells were either untreated or treated with 660 nM TSA. After maintenance at 38.5° C. for a further 2 days, extracts were made and β-galactosidase activities were measured. The extracts from untreated cells gave a value of 121 units (range 113-129), whereas extracts from TSA-treated cells gave a value of 806 unites (range 739-873), a stimulation of 6.7-fold. Enzyme activities in cells infected with in1382, which does not express pp71, were indistinguishable from those of mock-infected cells, irrespective of the presence of TSA.

This result shows that pp71 renders the quiescent genome responsive to the agent TSA. Photographs of monologues without treatment (a) or after treatment with 660 nM TSA for 2 days (b) are shown in FIG. 5. TSA treatment increased the number of positive cells and the intensity of straining.

### References

ACE, C. I., DALRYMPLE, M. A., RAMSAY, F. H., PRESTON, V. G., and PRESTON, C. M. (1988). Mutational analysis of the herpes simplex virus type 1 trans-inducing factor Vmw65. J. Gen. Virol. 69, 2595-2605.

ACE, C. I., McKEE, T. A., RYAN, J. M., CAMERON, J. M., and PRESTON, C. M. (1989). Construction and characterization of a herpes simplex virus type 1 mutant unable to transinduce immediate-early gene expression. J. Virol. 63, 2260-2269.

BRESNAHAN, W. I., and SHENK, T. (2000). UL82 virion protein activates expression of immediate early viral genes in human cytomegalovirus-infected cells. Proc. Nat. Acad. Sci. USA 97, 14506-143511.

DAVISON, M- J., PRESTON, V. G., and McGEOCH, D. J. (1984). Determination of the sequence alteration in the DNA of the herpes simplex virus type 1 temperature-sensitive mutant ts K. J. Gen. Virol. 65, 859-863.

EVERETT, R. D. (1987). A detailed mutational analysis of Vmw110, a trans-acting transcriptional activator encoded by herpes simplex virus type 1. EMBO J. 6, 2069-2076.

EVERETT, R. D., ORR. A. and PRESTON, C. M. (1998). A viral activator of gene expression functions via the ubiquitin-proteasome pathway. EMBO J. 17, 7161-7169.

HOMER, E. G., RINALDI, A., NICHOLL, M. J., and PRESTON, C. M. (1999). Activation of herpesvirus gene expression by the human cytomegalovirus protein pp71. J. Virol. 73, 8512-8518.

JAMIESON, D. R. S., ROBINSON, L. H., DAKSIS, J. I., NICHOLL, M. J., and PRESTON, C. M. (1995). Quiescent viral genomes in human fibroblasts after infection with herpes simplex virus Vmw65 mutants. J. Gen. Virol. 76, 1417-1431.

LIU, BAND STINSKI, M. F. (1992). Human cytomegalovirus contains a tegument protein that enhances transcription from promoters with upstream ATF and AP-1 cis-acting elements. Journal of Virology. 66, 4434-4444.

MACLEAN, C. A., EFSTATHIOU, S., ELLIOTT, M. L., JAMIESON, F. E., and McGEOCH, D. J. (1991). Investigation of herpes simplex virus type 1 genes encoding multiply inserted membrane proteins. J. Gen. Virol. 72, 897-906.

MARSHALL, K. R., LACHMANN, R. H., EFSTATHIOU, S., RINALDI, A., and PRESTON, C. M. (2000). Long-term transgene expression in mice infected with a herpes simplex virus type 1 mutant severely impaired for immediate-early gene expression. J. Virol. 74, 956-964.

PRESTON, C. M., and NICHOLL, M. J. (1997). Repression of gene expression upon infection of cells with herpes simplex virus type 1 mutants impaired for immediate early protein synthesis. J. Virol. 71, 7807-7813.

PRESTON, C. M., RINALDI, A., and NICHOLL, M. J. (1998). Herpes simplex virus type 1 immediate early gene expression is stimulated by inhibition of protein synthesis. Journal of General Virology 79, 117-124.

RINALDI, A., MARSHALL, K. R., and PRESTON, C. M. (1999). A non-cytotoxic herpes simplex virus vector which expresses Cre recombinase directs efficient site specific recombination. Virus Research 65, 11-20.

SAMANIEGO, L. A., NEIDERHISER, L., and DELUCA, N. A. (1998). Persistence and expression of the herpes simplex virus genome in the absence of immediate-early proteins. J. Virol. 72, 3307-3320.

SPAETE, R. R., and FRENKEL, N. (1982). The herpes simplex virus amplicon: a new eukaryotic defective-virus cloning-amplifying vector. Cell 30, 295-304.

1. A recombinant construct for expression of a transgene comprising:
(a) a first vector comprising a promoter operably linked to said transgene; and
(b) a gene for cytomegalovirus pp71 or homologues thereof in expressible form.

2. A recombinant construct as claimed in claim 1 where said first vector is a viral vector.

3. A recombinant construct as claimed in claim 2 wherein said first vector is a non-integrating viral vector.

4. A recombinant construct as claimed in claim 3 wherein said first vector is a Herpesvirus vector.

5. A recombinant construct as claimed in claim 4 wherein said first vector is obtained from or is a genetically manipulated version of HSV-1, HSV-2, HCMV or EBV.

6. A recombinant construct as claimed in claim 1 wherein the promoter of said first vector is a eukaryotic promoter.

7. A recombinant construct as claimed in claim 1 wherein said promoter of said first vector is the HSV-1 IE promoter.

8. A recombinant construct as claimed in claim 1 wherein the cytomegalovirus pp71 is the human, mouse, rat, chimpanzee, simian, equine or guinea pig pp71 or homologues thereof.

9. A recombinant construct as claimed in claim 1 wherein the gene for cytomegalovirus pp71 is the human gene.

10. A recombinant construct as claimed in claim 1 wherein expression of the transgene is regulatable by external factors.

11. A recombinant construct as claimed in claim 1 wherein the gene for cytomegalovirus pp71 forms part of and is expressed from a second vector.

12. A recombinant construct as claimed in claim 1 wherein the gene for cytomegalovirus pp71 is integrated into and is expressed from the host cell genome.

13. A recombinant construct as claimed in claim 1 wherein the gene for cytomegalovirus pp71 forms part of and is expressed from the first viral vector.

14. A recombinant construct as claimed in claim 13 wherein the transgene and the gene for cytomegalovirus pp71 are juxtaposed on the first viral vector.

15. A host cell transfected with a recombinant construct as claimed in claim 1.

16. A host cell as claimed in claim 15 which is a mammalian host cell.

17. A method of maintaining expression of a transgene in a vector, said method comprising introducing the gene for cytomegalovirus pp71 or homologues thereof into the vector while maintaining the functionality of any cis-acting sequence.

18. The method as claimed in claim 17 wherein said vector is a non-integrating viral vector.

19. The method as claimed in claim 18 wherein said vector is a Herpesvirus vector.

20. The method as claimed in claim 19 wherein said vector is obtained from or is a genetically manipulated version of HSV-1, HSV-2, HCMV or EBV.

21. The method as claimed in claim 17 wherein the cytomegalovirus pp71 is the human, mouse, rat, chimpanzee, simian, equine or guinea pig pp71 or homologues thereof.

22. The method as claimed in claim 21 wherein the cytomegalovirus pp71 is human pp71 or homologues thereof.

23. A method of maintaining expression of a transgene in an expression vector, said method comprising providing cytomegalovirus pp71 during expression of said transgene.

24. The method as claimed in claim 23 wherein said vector is a non-integrating viral vector.

25. The method as claimed in claim 24 wherein said vector is a Herpesvirus vector.

26. The method as claimed in claim 25 wherein said vector is obtained from or is a genetically manipulated version of HSV-1, HSV-2, HCMV or EBV.

27. The method as claimed in claim 23 wherein the cytomegalovirus pp71 is the human, mouse, rat, chimpanzee, simian, equine or guinea pig pp71 or homologues thereof.

28. The method as claimed in claim 23 wherein the cytomegalovirus pp71 is human pp71 or homologues thereof.

29. A method of treating a patient having a disease or disorder, said method comprising introducing to said patient recombinant constructs as claimed in claim 1, wherein said first vector comprises a transgene encoding RNA or protein of therapeutic value for said disease or disorder.

30. The method is claimed in claim 29 wherein said disease or disorder is a neurological disease or disorder.

31. (canceled)

32. A method of producing a target protein or peptide, said method comprising providing a first vector comprising a promoter operably linked to a transgene encoding the target protein or peptide and wherein during expression of said transgene the transgene is exposed to cytomegalovirus pp71 or a homologue thereof.
